(19) United States
(12) Patent Application Publication
(10) Pub. No.: US 2014/0151432 A1
(43) Pub. Date: Jun. 5, 2014

(54) Device and Method for Fastening Connective Tissue

(71) Applicant: Ronald W. Weaver, JR., Atlanta, GA (US)

(72) Inventor: Ronald W. Weaver, JR., Atlanta, GA (US)

(21) Appl. No.: 13/740,993

(22) Filed: Jan. 14, 2013

Related U.S. Application Data
(60) Provisional application No. 61/732,600, filed on Dec. 3, 2012.

(51) Int. Cl. A61B 17/068

(52) U.S. Cl. CPC A61B 17/068
USPC 227/176.1

(57) ABSTRACT

A stapler delivers a fastener so that the legs of the fastener penetrate two abutting portions of fascia from the inside out so that, after penetration, the legs of the fastener are outside the body. The stapler subsequently positions a retainer over the ends of the fastener in order to hold the fascia and fastener in place. Once the retainer is properly positioned, the stapler may trim and capture the ends of the fastener that protrude beyond the retainer to avoid patient discomfort, injury or post-operative complications. A wide variety of fastener and retainer combinations are presented for use in differing environments and conditions.

### PRIORITY STATEMENT UNDER 35 U.S.C. §119 & 37 C.F.R. §1.78

This non-provisional application claims priority based upon prior U.S. Provisional Patent Application Ser. No. 61/732,600 filed Dec. 3, 2012 in the name of Ronald W. Weaver, Jr. entitled “Device and Method for Fastening Connective Tissue,” the disclosure of which is incorporated herein in its entirety by reference as if fully set forth herein.

### BACKGROUND OF THE INVENTION

This invention relates to an improved method and system for joining, by stapling, the edges of fascia so as to maintain the edges of a wound in close approximation to facilitate healing.

One of the new developments in surgery in recent years has been the replacement of conventional thread sutures for closing wounds or incisions in living tissue or a patient with of a surgical staple. Surgical staplers typically consist of a skin stapler and metal staples. Such use of skin staplers and metal staples have clearly and successfully demonstrated a reduction in suturing time and, thus, overall operating time during which the patient must remain in the operating room under anesthesia.

There are a wide variety of surgical staplers on the market today, such as manual and gas powered instruments, but few are widely accepted by doctors for a number of reasons, such as poor visibility of wound site, cost, reliability, bulkiness, lack of familiarity with the product, clumsiness and/or difficulty in use due to mechanical complexity. In addition, surgical staplers known in the art are configured to have the staple positioned outside the body prior to deployment with the legs of the staple positioned such that the teeth, the sharpened portion of the leg of the fastener, penetrate into the body and are then crimped. As a result, after deployment the teeth are positioned inside the body which may result in patient discomfort, further injury to the patient, or post-operative complications, particularly if the legs of the staple are not properly crimped during application or become pulled apart thereafter.

Therefore, a need exists for a reliable surgical device for use during surgical procedures that can be employed so that the teeth of the fastener are outside the patient's body after deployment. It would also be beneficial if the fastener could be secured in place through the use of a retainer to alleviate concerns regarding improper crimping. In addition, it would be beneficial if the surgical device could perform multiple tasks, including delivering the fastener through the fascia, applying the retainer over the legs of the fastener, clipping any excess portion of the legs of the fastener that protrude beyond the retainer, and capturing the clipped portion of the legs within or without the surgical device to prevent them from contaminating the surgical site.

### SUMMARY OF THE INVENTION

The present invention relates generally to the fastening of abutting portions of fascia and, more particularly, to post-surgical fascial closure. In one embodiment, a stapler drives legs of a fastener through two abutting portions of fascia tissue and then connects a retainer to the ends of the fastener protruding through the fascia in order to hold the fascia and fastener in place. After the retainer has been placed over the legs of the fastener, the stapler may clip and capture the ends of the fastener that protrude beyond the retainer to avoid patient discomfort, injury or post-operative complications that may result from abrasion against the exposed leg portion.

In another embodiment, a fastener is configured with a notch below the teeth on each leg of the fastener and a retainer is configured with two openings through which the legs may pass. In use, the teeth of the fastener penetrate the fascia and then pass through the openings in the retainer. The sides of the opening are approximately the same dimension as the notches in the legs of the fastener such that the sides of the opening engage into the notches to secure the fastener in place.

The foregoing has outlined rather broadly certain aspects of the present invention in order that the detailed description of the invention that follows may better be understood. Additional features and advantages of the invention will be described hereinafter which form the subject of the claims of the invention. It should be appreciated by those skilled in the art that the conception and specific embodiment disclosed may be readily utilized as a basis for modifying or designing other structures or processes for carrying out the same purposes of the present invention. It should also be realized by those skilled in the art that such equivalent constructions do not depart from the spirit and scope of the invention as set forth in the appended claims.

### DESCRIPTION OF THE DRAWINGS

For a more complete understanding of the present invention, and the advantages thereof, reference is now made to the following descriptions taken in conjunction with the accompanying drawings, in which:

FIGS. 1a and 1b show front and side views, respectively, of one embodiment of a single fastener of the present invention;

FIGS. 1c and 1d show front and side views, respectively, of one embodiment of a fastener cartridge of the present invention;

FIGS. 1e and 1f show front and side views, respectively, of a retainer cartridge of the present invention;

FIG. 2a shows a side view of one embodiment of a fastener of the present invention;

FIG. 2b shows a side view of one embodiment of a retainer of the present invention;

FIG. 2c shows a top view of one embodiment of a retainer connected to a fastener of the present invention;

FIG. 2d shows a side view of one embodiment of a retainer of the present invention connected to a fastener of the present invention;

FIGS. 3a and 3b show a cross sectional side view and a cross-sectional top view, respectively, of a stapler of one embodiment of the present invention;

FIG. 4 and FIG. 5 show a side view and an isometric view, respectively, of a stapler in one embodiment of the present invention;

Referring now to FIG. 6 and FIG. 7 which show a side view and an isometric view, respectively, of a stapler in another embodiment of the present invention;

Referring now to FIG. 8 and FIG. 9 which show a left isometric view and a right isometric view, respectively, of a fastener on one embodiment of the present invention.

FIG. 10 shows an isometric view of yet another embodiment of a fastener of the present invention;

FIG. 11 and FIG. 12 show a left isometric vies and a right isometric view, respectively, of another embodiment of a fastener of the present invention;

FIG. 13, FIG. 14, and FIG. 15 show one embodiment of a staple of the present invention in a lower, upper and isometric, respectively, view of a fastener of the present invention;

FIG. 16 and FIG. 17 shows a left isometric view and a right isometric view, respectively, of a retainer of one embodiment of the present invention; and

Referring now to FIG. 18 which shows an isometric view of one embodiment of a fastener of the present invention connected to a retainer.

### DETAILED DESCRIPTION OF THE EMBODIMENTS

The present invention is directed to a surgical stapler for use in joining the skin or fascia of a patient after surgery. The configuration and use of the presently preferred embodiments are discussed in detail below. It should be appreciated, however, that the present invention provides many applicable inventive concepts that can be embodied in a wide variety of contexts other than joining the skin or fascia of a patient. Accordingly, the specific embodiments discussed are merely illustrative of specific ways to make and use the invention, and do not limit the scope of the invention. In addition, the following terms shall have the associated meaning when used herein:

“fascia” means and includes skin and the structure of connective tissue that surrounds muscles, groups of muscles, blood vessels, and nerves, binding some structures together, while permitting others to slide smoothly over each other, including superficial fascia, deep fascia, and visceral fascia; and

“fastener” means any generally “U” or “D” shaped object capable of penetrating fascia.

In one embodiment of the present invention, a stapler may connect fascia tissue together. For example, a stapler may press a fastener through two abutting portions of fascia and connect a retainer over the ends of the fastener in order to hold the fascia and fastener in place. After the retainer has been placed over the ends of the fastener, the stapler may clip the ends of the fastener to avoid patient discomfort, injury or post-operative complications.

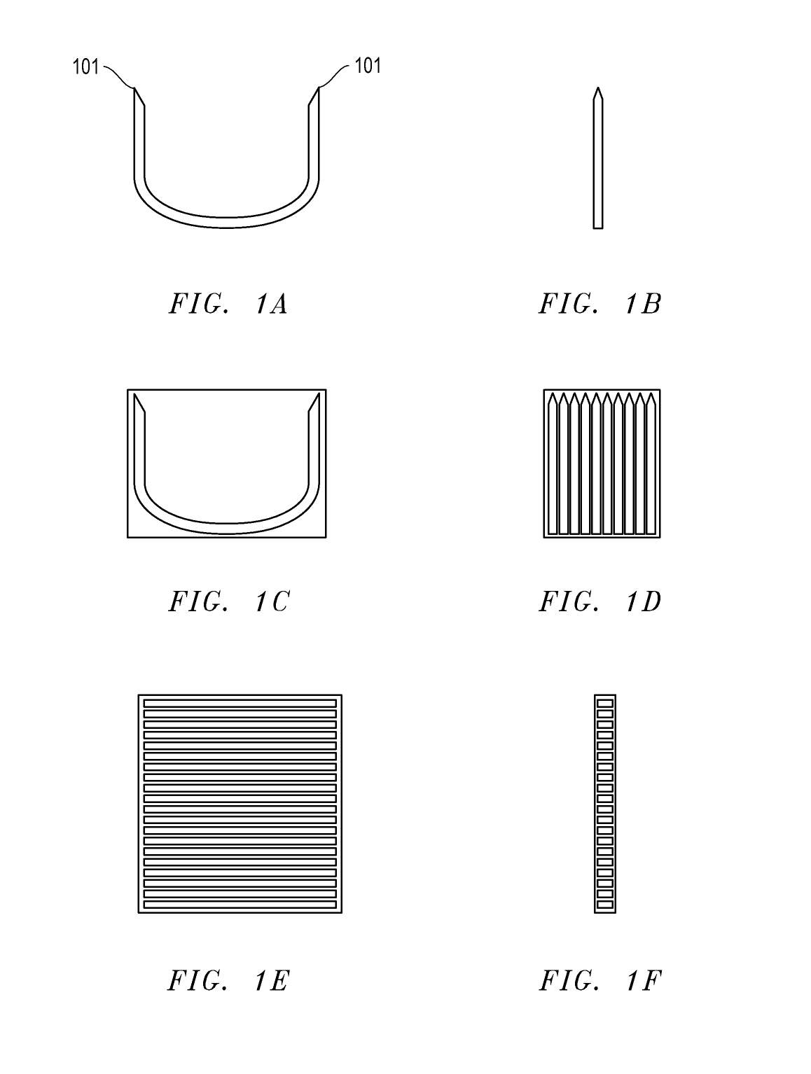

FIG. 1a and FIG. 1b show a front view and a side view, respectively, of a single fastener in one embodiment of the present invention. In this embodiment, the fastener is generally U-shaped. The ends 101 of the fastener are sharp so that they may penetrate through connective tissue such as fascia or muscle.

FIG. 1c and FIG. 1d show a front view and a side view, respectively, of a fastener cartridge in one embodiment of the present invention. In this embodiment, the fastener cartridge is shown to contain ten fasteners.

FIG. 1e and FIG. 1f show a front view and a side view, respectively, of a retainer cartridge in one embodiment of the present invention. In this embodiment, the retainer cartridge is shown to contain twenty retainers. As explained in more detail below, a retainer may be configured to fit over the legs of a fastener. Any form of cutter or clipper known in the art may be used to clip the legs of a fastener to the extent that the legs extend beyond a retainer that is connected to the fastener. In some embodiments, a portion of the cutter or clipper may be configured so that any clipped legs of fasteners will be contained within an enclosed space with or without the cutter or clipper.

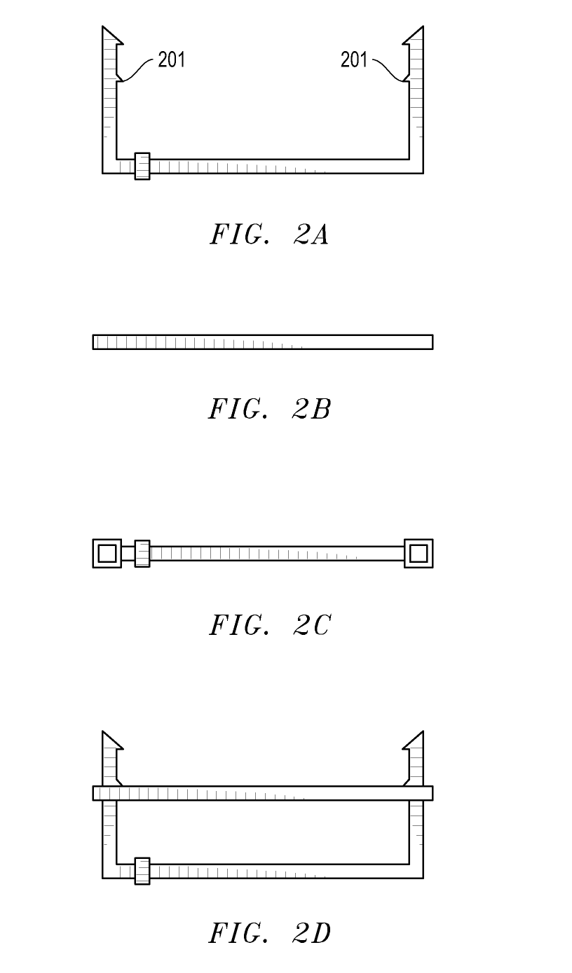

FIG. 2a shows a side view of a fastener in one embodiment of the present invention. In this embodiment, the fastener is approximately 2 centimeters in height and approximately 4 centimeters in length. In this embodiment, the fastener includes protrusions on the inside surface of the arms of the fastener. The protrusions may be used to secure a retainer to the fastener. In this embodiment, the notches are located approximately 1.75 centimeters from the base of the fastener, but they may be placed in other locations according to the requirements of the application.

FIG. 2b and FIG. 2c show a side view and a top view, respectively, of a retainer in one embodiment of the present invention. FIG. 2d shows a side view of a retainer connected to a fastener in one embodiment of the present invention. In this embodiment, the ends of the fastener that extend beyond the retainer may be clipped after the fastener is connected to fascia and a retainer is connected to the fastener. For example, clipping the ends of the fastener may prevent the ends from continuing to perforate tissue of the patient which could lead to patient discomfort, injury or post-operative complications.

The fasteners described herein may be applied singly in succession or a number may be applied simultaneously. The legs of the fastener are driven through one side of the tissue to be fastened and the retainer interlocks with the prongs of the fastener on the other side of the tissue to hold the entire fastener structure in place.

Fasteners of the present invention may be made of metal, such as tantalum or stainless steel, which are inert or, alternatively, of magnesium, which is gradually absorbed by the body. Fasteners may also be non-metallic which, in some cases, may have certain advantages over metal fasteners such as, for example, not scattering x-rays during subsequent imaging.

Also, fasteners of the present invention are not limited to specific widths, heights or depths. Depending on the application, the legs of the fastener may be longer or shorter, the crown of the fastener may be longer, shorter curved or straight, the teeth on the end of the legs may be sharp or blunted, etc. Also, the cross sectional shape of the legs and crown can be selected as required or desired to be round, oval, quadrangular, or any other shape known in the art. Once the shape of the fastener is determined, a stapler of the present invention can be configured with operational dimensions capable of supporting the fastener or magazines containing multiple fasteners.

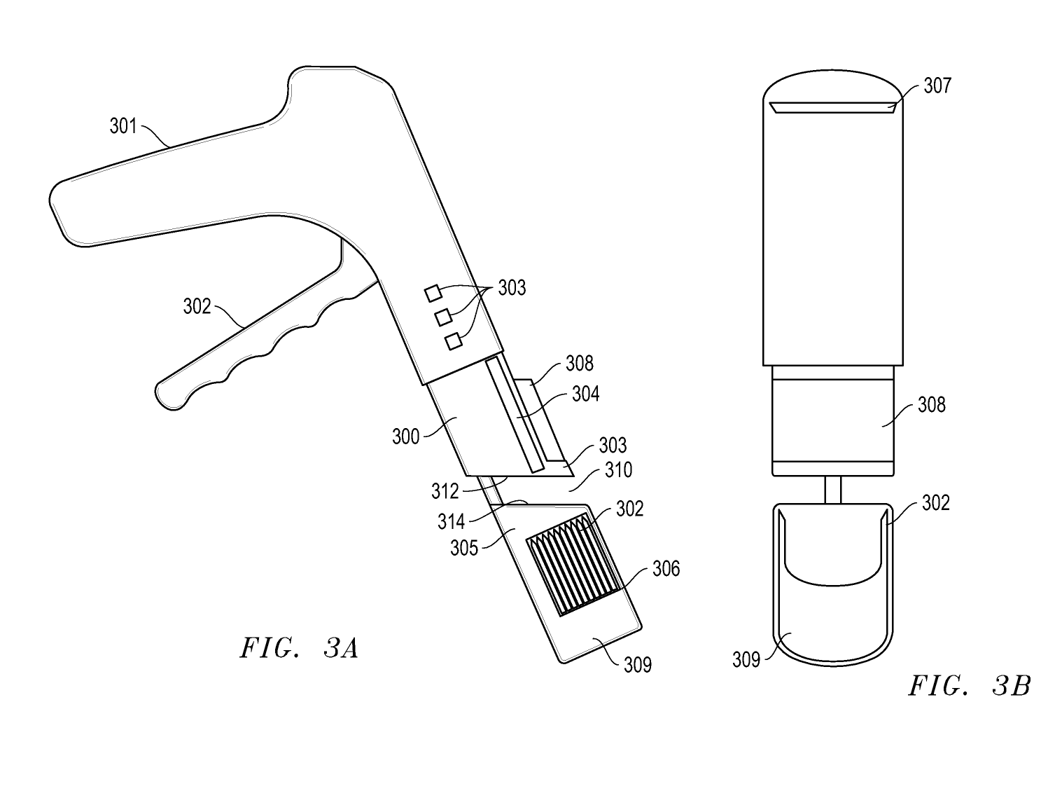

Referring now to FIG. 3a and FIG. 3b which show a cross-sectional side view and cross-sectional top view, respectively, of a stapler in one embodiment of the present invention. In this embodiment, the stapler includes a gap 310 into which the fascia or skin to be connected is placed, the carriage 305 being positioned under the fascia and the base 300 being positioned over the top of the fascia. More specifically, the distal face 312 of base 300 is positioned on the top of the fascia and the proximal face 314 of carriage 305 is placed under the fascia.

The stapler of this embodiment further includes a handle 301 and grip 302. In use, a caregiver may pull the grip 302 towards the handle 301, while holding the handle 301 in the palm of her hand and pulling the grip 302 with her fingers on the same hand. For example, by pulling the grip 302 towards the handle 301, carriage 305 will move towards the base 300, causing the ram 309 to press a fastener 302 from the fastener cartridge 306 through the fascia tissue and into a retainer 303 located in or adjacent to retainer cartridge 304, thereby securing the retainer 303 over the ends of the fastener 302. Ram 309 may push staples mechanically, hydraulically, or by any other means known in the art.

In one embodiment, the stapler may also include clipper mechanism 308 to remove the ends of the fastener 302 that extend beyond the retainer 303. In addition, the stapler may include a retainer cartridge slot 307 for inserting the retainer cartridge 304 into the stapler.

In one embodiment, the stapler may include openings or depressions 303 that can be used to widen or narrow the gap 310 depending on the thickness of the fascia being fastened. For example, a mechanical interface on base 300 may interlock with the proximal-most opening or depression 303, thereby creating the widest gap 310 possible. As the mechanical interface on base 300 moves into more distally positioned openings or depressions 303, gap 310 narrows.

As will be recognized by those skilled in the art, the stapler of the present invention may, in different embodiments, be disposable after a single use or, alternatively, may be autoclaved for multiple uses.

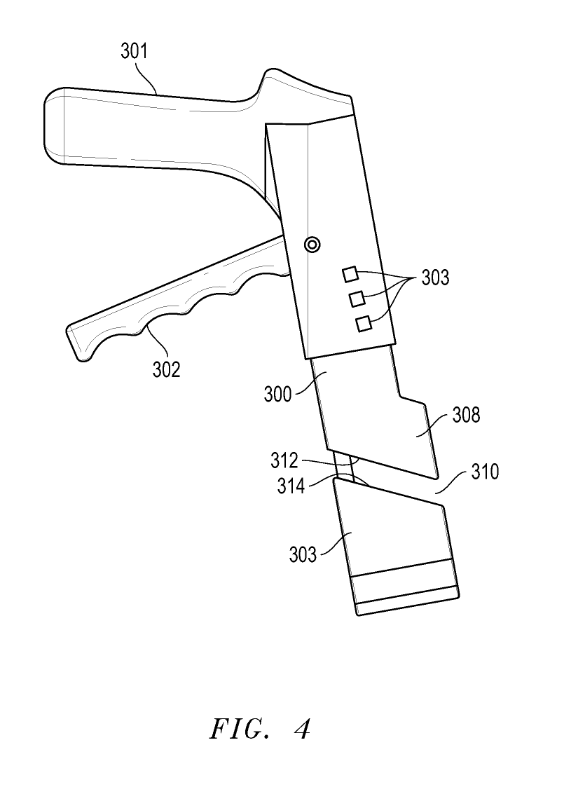

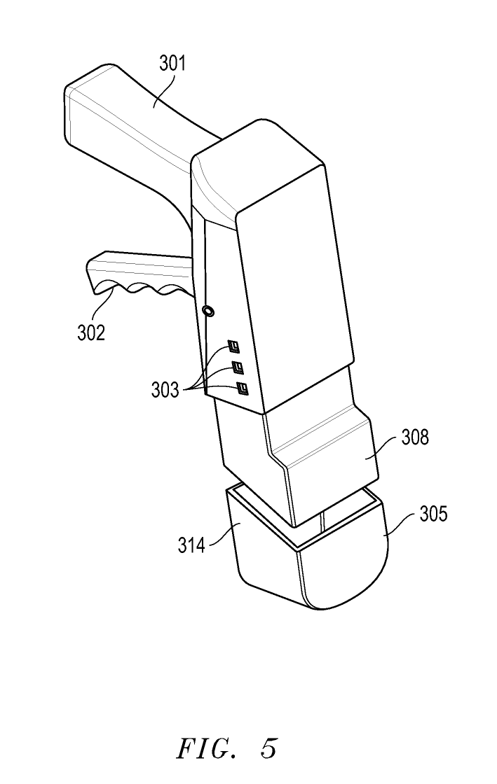

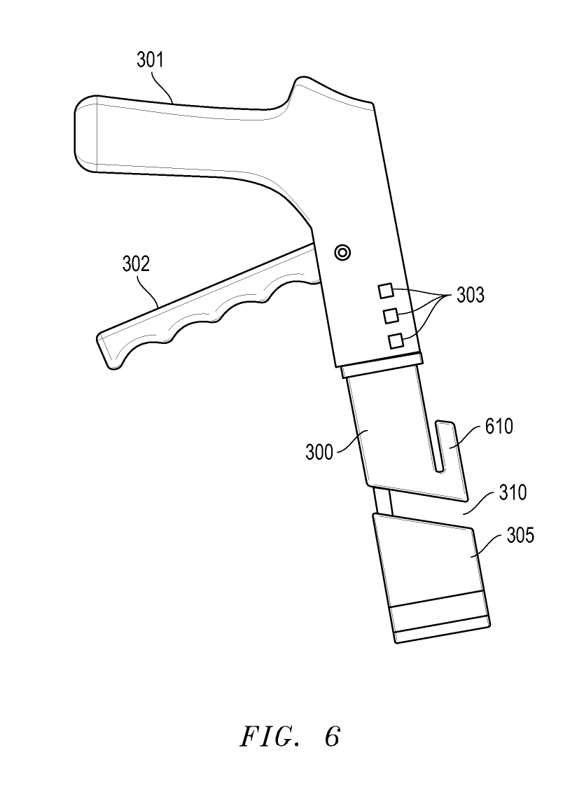

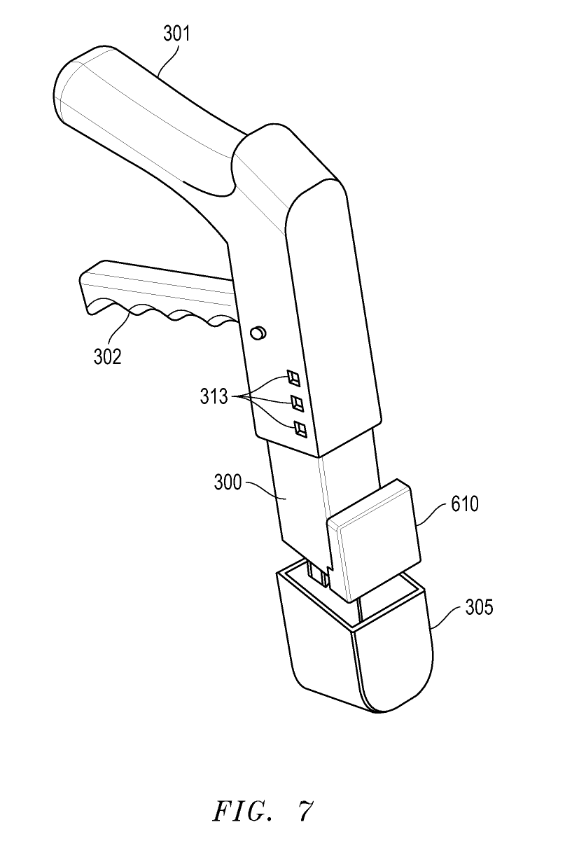

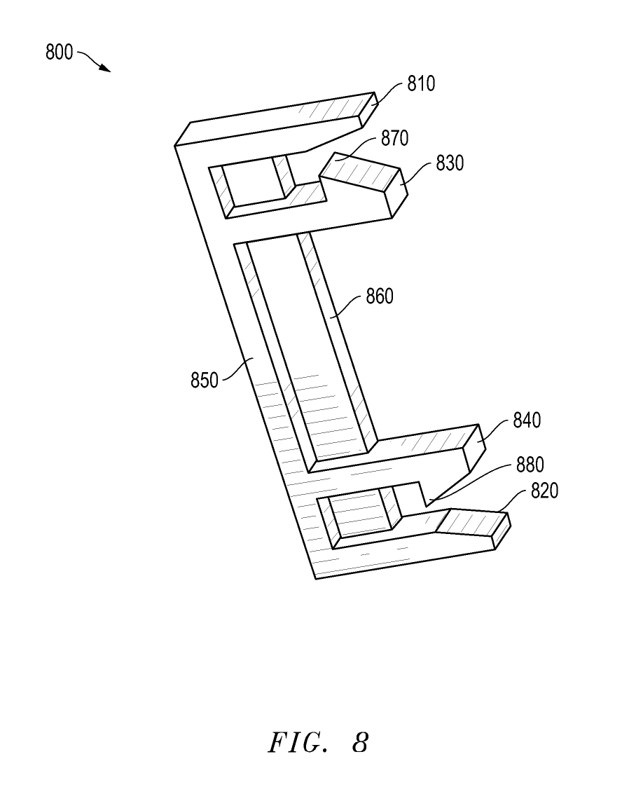

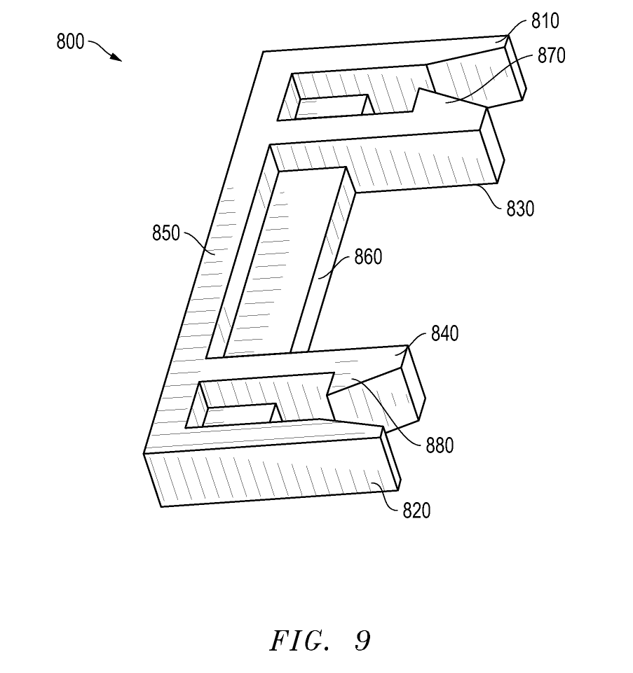

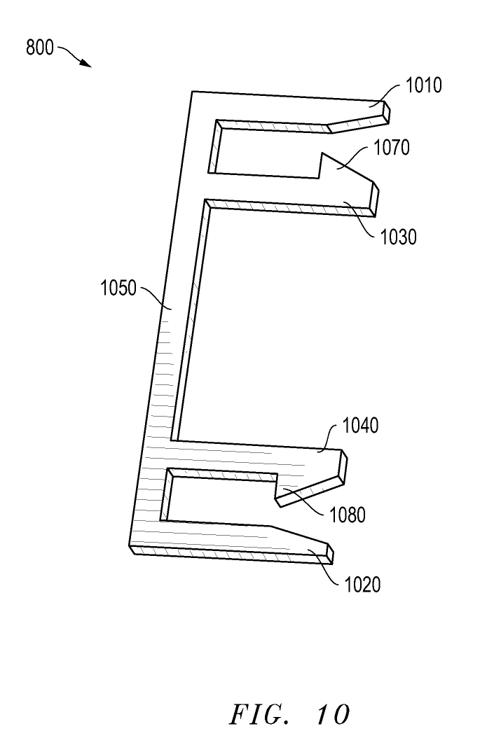

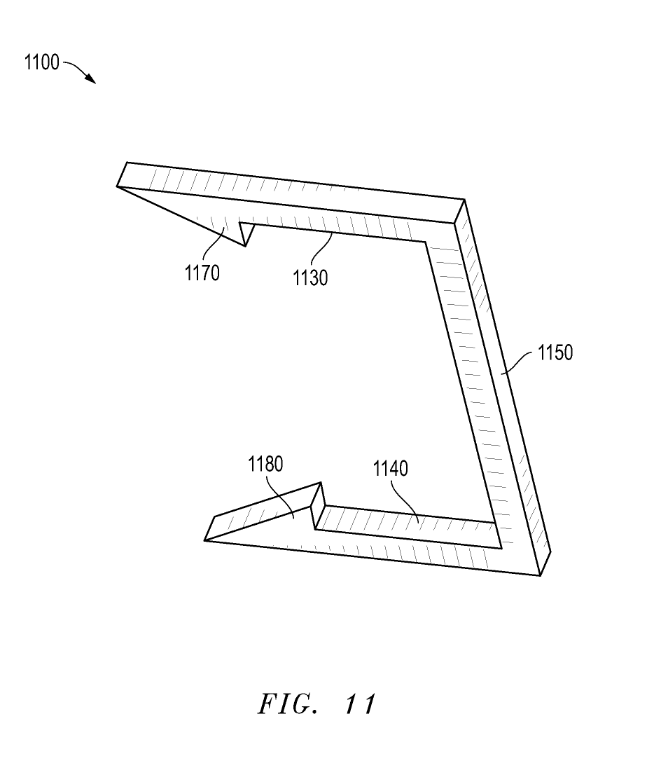

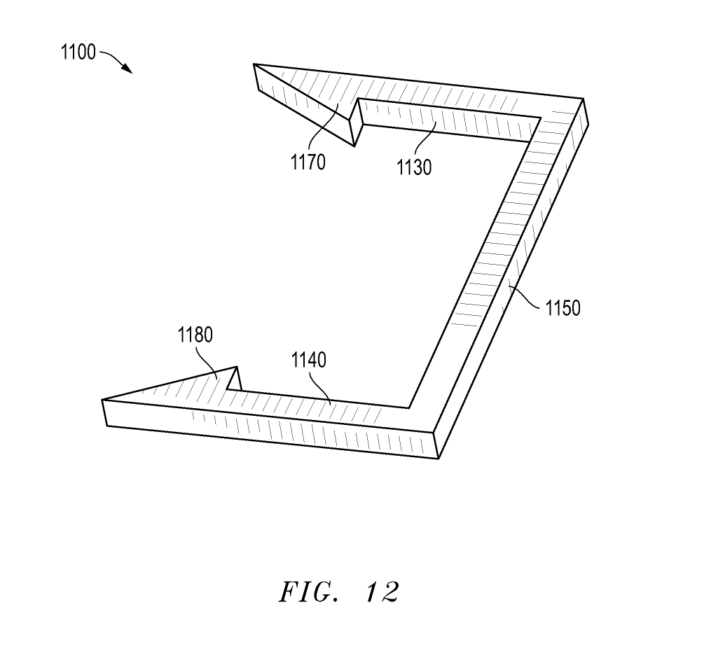

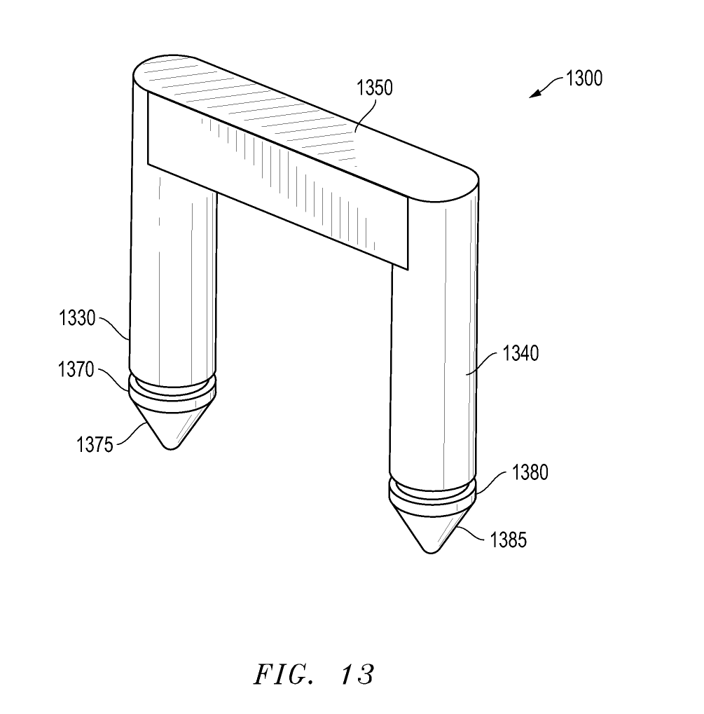

FIG. 4 and FIG. 5 show a side view and an isometric view, respectively, of a stapler in one embodiment of the present invention without cross-sectioning. Aspects of the stapler are consistently labeled with the same reference numbers as in FIG. 3a and FIG. 3b.

Referring now to FIG. 6 and FIG. 7 which show a side view and an isometric view of a stapler in another embodiment of the present invention. Again, aspects of the stapler are consistently labeled with the same reference numbers as in FIG. 3a and FIG. 3b. In this embodiment, however, the stapler includes a retainer cartridge 610 that is configured to be removable from, and replaceable on, the stapler. For example, cartridge 610 may be configured with mechanical interlocks such that it snaps on and off of the stapler.

Referring now to FIG. 8 and FIG. 9 which show a left isometric view and a right isometric view, respectively, of a fastener 800 of one embodiment of the present invention. This fastener is configured with two outer legs 810 and 820, two inner legs 830 and 840, and crown 850. Although in this embodiment, the teeth of legs 810, 820, 830 and 840 are blunted, the teeth of legs 810, 820, 830 and 840 may be sharpened or blunted as required for the application. Fastener 800 also includes a stabilizer 860 to assist in maintaining the alignment of legs 810, 820, 830 and 840, particularly as they are guided into the retainer (not shown). Inner legs 830 and 840 include protrusions 870 and 880 for locking the retainer in place during use.

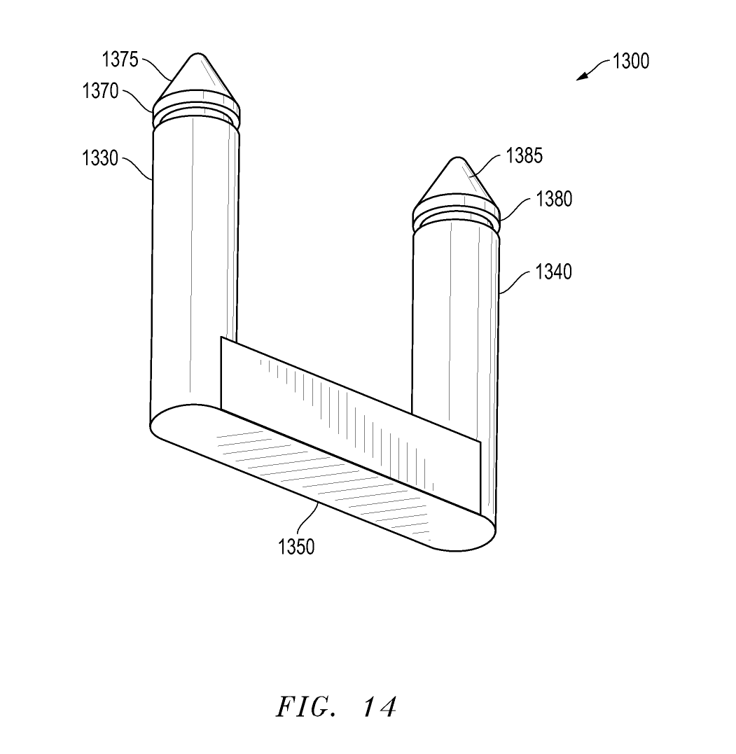

FIG. 10 shows an isometric view of yet another embodiment of a fastener 1000 of the present invention. In this configuration, fastener 1000 is similarly configured to fastener 800 except that fastener 1000 may be narrower than fastener 800 and fastener 1000 does not have a stabilizer. The configuration of fastener 1000 may be desirable in some applications due to its lower cost.

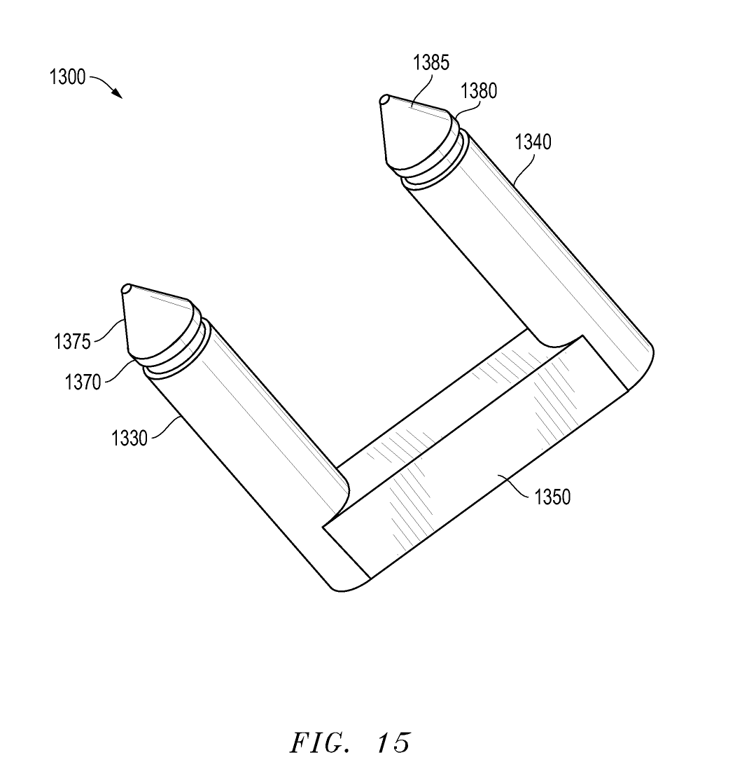

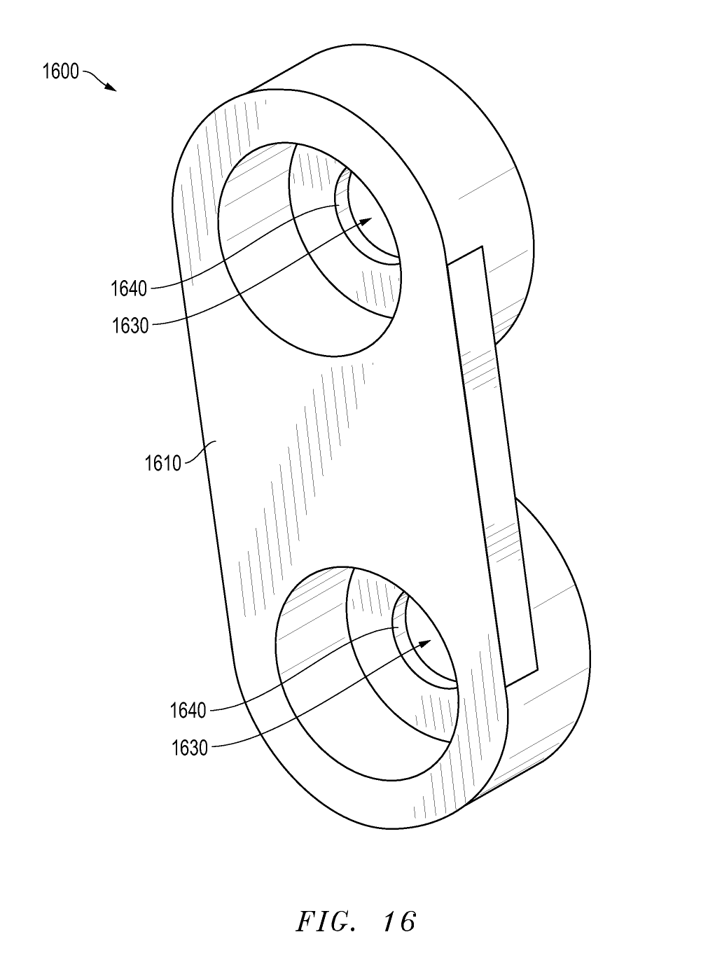

FIG. 11 and FIG. 12 show a left isometric vies and a right isometric view, respectively, of another embodiment of a fastener 1100 of the present invention. In this embodiment, fastener 1100 includes legs 1130 and 1140 and crown 1150. Each of the legs 1130 and 1140 include protrusions 1170 and 1180 for use in locking the retainer (not shown) in place during use.

FIG. 13, FIG. 14, and FIG. 15 show one embodiment of a staple of the present invention in a lower, upper and isometric views, respectively, of a fastener 1300 of the present invention. In this embodiment, fastener 1300 has legs 1330 and 1340 that are round in cross section and crown 1350 that is quadrangular in cross section. Each of the legs 1330 and 1340 has a conical tooth 1375 and 1385, respectively, on the end. Each of the legs 1330 and 1340 each also has a notch 1370 and 1380, respectively, into which a retainer may be positioned during use.

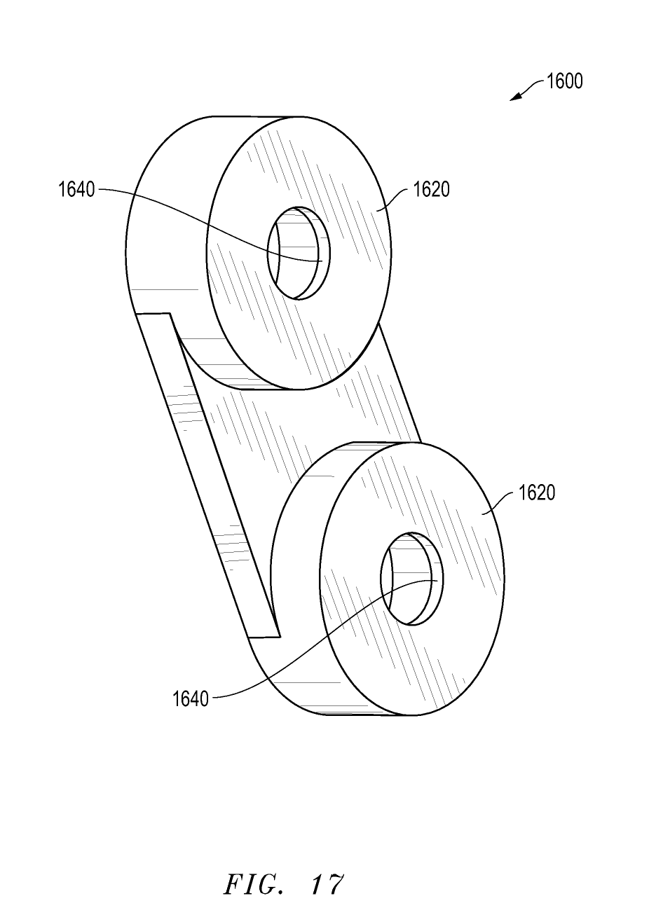

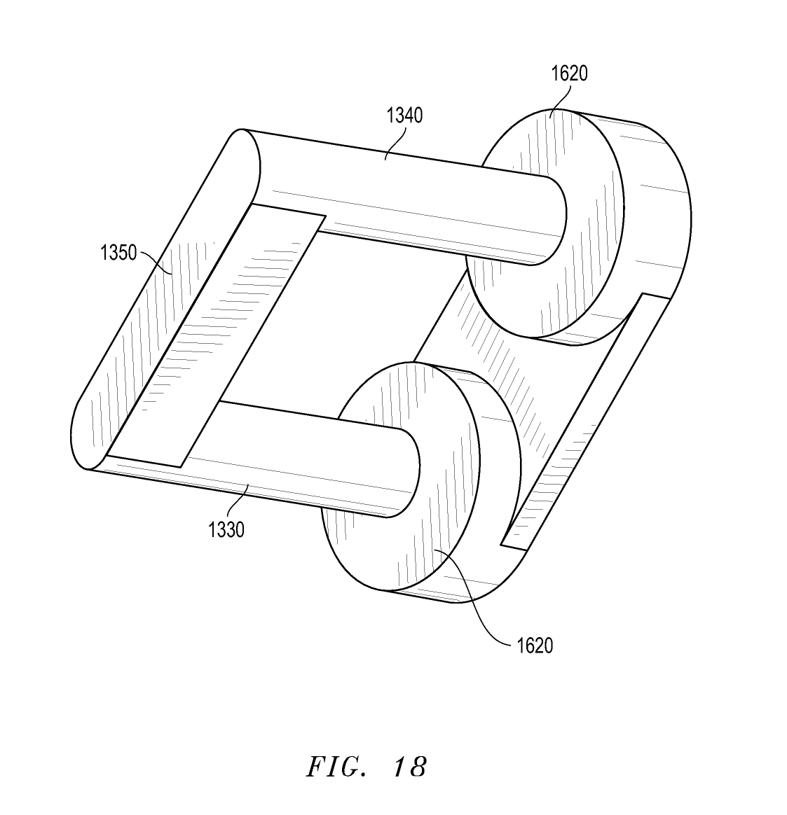

FIG. 16 and FIG. 17 shows a left isometric view and a right isometric view, respectively, of a retainer 1600 of one embodiment of the present invention. Retainer 1600 includes a lower surface 1610 and two raised upper surfaces 1620. The upper surfaces 1620 are each round in cross section, but they may be any shape known in the art. Each of the raised upper surfaces includes an opening 1630 through which fastener legs (not shown) may pass.

Referring now to FIG. 18 which shows an isometric view of one embodiment of a fastener 1300 of the present invention connected to a retainer 1600 of the present invention. In use, a fastener of the present invention, such as fastener 1300 can be applied through the use of a stapler or other means to pass the teeth 1375 and 1385 of the fastener through the fascia (not shown) and into the opening of the retainer, such as opening 1630 of retainer 1600. To secure fastener 1300 in place, the edge 1640 of the opening 1630 is positioned in notch 1370 and 1380. In this configuration, teeth 1375 and 1385 are enclosed within the raised portion of the retainer so as to minimize the risk that the teeth will further penetrate fascia or nearby organs.

While the present system and method has been disclosed according to the preferred embodiment of the invention, those of ordinary skill in the art will understand that other embodiments have also been enabled. Even though the foregoing discussion has focused on particular embodiments, it is understood that other configurations are contemplated. In particular, even though the expressions “in one embodiment” or “in another embodiment” are used herein, these phrases are meant to generally reference embodiment possibilities and are not intended to limit the invention to those particular embodiment configurations. These terms may reference the same or different embodiments, and unless indicated otherwise, are combinable into aggregate embodiments. The terms “a”, “an” and “the” mean “one or more” unless expressly specified otherwise. The term “connected” means “communicatively connected” unless otherwise defined.

When a single embodiment is described herein, it will be readily apparent that more than one embodiment may be used in place of a single embodiment. Similarly, where more than one embodiment is described herein, it will be readily apparent that a single embodiment may be substituted for that one device.

In light of the wide variety of possible connective tissue fastening devices and methods available, the detailed embodiments are intended to be illustrative only and should not be taken as limiting the scope of the invention. Rather, what is claimed as the invention is all such modifications as may come within the spirit and scope of the following claims and equivalents thereto.

None of the description in this specification should be read as implying that any particular element, step or function is an essential element which must be included in the claim scope. The scope of the patented subject matter is defined only by the allowed claims and their equivalents. Unless explicitly recited, other aspects of the present invention as described in this specification do not limit the scope of the claims.

I claim:

1. A method for connecting fascia comprising:
positioning two abutting portions of fascia in a gap between a carriage with a fastener cartridge positioned therein and a base with a retainer cartridge positioned therein, wherein said carriage is placed inside a body cavity and said base is positioned outside said body cavity;
moving said carriage towards said base wherein a fastener is dislodged from said fastener cartridge, legs of said fastener pass through said fascia and through at least one opening in a retainer from said retainer cartridge.

2. The method of claim 1, wherein at least a portion of said legs that protrude beyond said opening in said retainer after insertion therethrough is removed.

3. The method of claim 1, wherein at least a portion of said legs that protrude beyond said retainer are removed and captured after removal.

4. The method of claim 1, wherein said legs include a notch configured to interlock with an edge of said opening in said retainer.

5. The method of claim 1, wherein said legs include protrusions configured to secure said legs within said retainer after said legs have passed through said opening in said retainer.

6. The method of claim 1, wherein said fastener is made of a material that may be absorbed into a human body.

7. A system for connecting fascia comprising:
a carriage with a fastener cartridge positioned therein and a base with a retainer cartridge positioned therein, wherein said carriage is configured for placement inside a body cavity and said base is configured for placement outside said body cavity;
a gap between said base and said carriage configured for the placement of two abutting portions of fascia therein, said base and cartridge configured so that, as said carriage moves towards said base, a fastener is dislodged from said fastener cartridge and said legs of said fastener are pushed through said fascia and through at least one opening in a retainer from said retainer cartridge.

8. The system of claim 7, wherein at least a portion of said legs that protrude beyond said opening in said retainer after insertion therethrough is removed.

9. The system of claim 7, wherein at least a portion of said legs that protrude beyond said opening in said retainer after insertion therethrough is removed and captured after removal.

10. The system of claim 7, wherein said legs include a notch configured to interlock with an edge of said opening in said retainer.

11. The system of claim 7, wherein said legs include protrusions configured to secure said legs within said retainer after said legs have passed through said opening in said retainer.

12. The system of claim 7, wherein said fastener is made of a material that may be absorbed into a human body.

13. A device for connecting two abutting portions of fascia comprising:
a carriage with a fastener cartridge positioned therein and a base with a retainer cartridge positioned therein, wherein said carriage is configured for placement inside a body cavity and said base is configured for placement outside said body cavity;
a gap between said base and said carriage configured for the placement of two abutting portions of fascia therein;
means for moving said carriage towards said base, causing a fastener to dislodge from said fastener cartridge and pushing legs of said fastener through said fascia and through at least one opening in a retainer from said retainer cartridge.

14. The device of claim 13, further including means for removing at least a portion of said legs that protrude beyond said opening in said retainer after insertion therethrough.

15. The device of claim 13, further including means for removing and capturing at least a portion of said legs that protrude beyond said opening in said retainer after insertion therethrough.

16. The device of claim 13, wherein said legs include a notch configured to interlock with an edge of said opening in said retainer.

17. The device of claim 13, wherein said legs include protrusions configured to secure said legs within said retainer after said legs have passed through said opening in said retainer.

18. The device of claim 13, wherein said fastener is made of a material that may be absorbed into a human body.
